(19) United States
(12) Patent Application Publication
(10) Pub. No.: US 2007/0142337 A1
(43) Pub. Date: Jun. 21, 2007

(54) Treatment of inflammation and organ dysfunction

(75) Inventors: Geert W. Schmid-Schonbein, Del Mar, CA (US); Frank A. Delano, San Diego, CA (US)

Correspondence Address: MOAZZAM & ASSOCIATES, LLC, 7601 LEWINSVILLE ROAD, SUITE 304, MCLEAN, VA, 22102, US

(73) Assignee: The Regents of the University of California

(21) Appl. No.: 11/638,432

(22) Filed: Dec. 14, 2006

Related U.S. Application Data
(60) Provisional application No. 60/750,356, filed on Dec. 15, 2005.

(51) Int. Cl. A61K 31/65

(52) U.S. Cl. 514/152

(57) ABSTRACT

Compositions and methods are disclosed for decreasing the amount and activity of matrix-degrading metalloproteinases (MMPs) in and around cells. The compositions and methods are useful for treating and/or preventing symptoms of diseases including but not limited to Hypertension, Type II Diabetes, and Metabolic Syndrome X. Such treatment is effected by normalizing the blood plasma's protease activity, reducing blood pressure, preventing membrane receptor cleavage, and reducing the levels of insulin resistance and oxygen free radicals in the blood stream.

This application claims priority to U.S. Provisional Patent Application Ser. No. 60/750,356, filed Dec. 15, 2005, the content of which is hereby incorporated by reference in its entirety into this disclosure.

### GOVERNMENT INTERESTS

This invention was made with U.S. Government support under National Institute of Health (NIH) Grant No. HL10881. The government has certain rights in this invention.

### BACKGROUND OF THE INVENTION

1. Field of the Invention

The present invention relates generally to the treatment of inflammatory symptoms and organ dysfunction. More particularly, the present invention relates to the treatment of inflammation and organ dysfunction by targeting matrix-degrading metalloproteinases.

2. Background of the Invention

An increasing number of cardiovascular complications have shown to be accompanied by markers of inflammation. These conditions include not only acute complications, such as cardiac or cerebral ischemia, atherosclerosis, pain, or the metabolic syndrome, but also chronic degenerative conditions such as ocular disease, venous disease, arterial hypertension, diabetes, arthritis, as well as the process of aging. The inflammatory cascade involves a sequence of stereotypic events that can be observed in all organs and in many situations is observed in the microcirculation years before clinical manifestations.

It has become evident in recent work that interventions against individual steps in the inflammatory cascade (e.g., blockade of oxygen free radicals, cytokines, membrane adhesion molecules, blockade of the complement and thrombotic cascades or production of lipid mediators) leads to a mixed level of success, in some cases despite preclinical evidence suggesting that the inflammatory process can be attenuated.

About a decade ago, a different approach was sought to analyze the inflammatory cascade. The basic idea was instead of looking exclusively at individual steps and/or markers for the inflammatory cascade, to develop an approach that serves to identify trigger mechanisms for inflammation. This approach has lead to a fundamental new understanding of the origin of inflammation in acute situations.

An important consideration to control the level of inflammation is to identify molecular mechanisms that can trigger an inflammatory reaction. To achieve this, it is most useful to detect inflammation at an early stage. One of the ways is to study the level of cell activation in the microcirculation. Besides the study of endothelial cells and mast cells in the tissue, a number of techniques have been developed to study cell activation by use of circulating leukocytes or platelets. Markers of leukocyte activation include pseudopod formation (by actin polymerization), free radical production (detectable by use of biochemical indicators and by electrochemical sensors), expression of membrane adhesion molecules or detection of the level of degranulation. Other markers are also possible.

These studies showed that there are a large number of mechanisms to activate cells and stimulate an inflammatory cascade. Because of the large number of mechanisms that may be exhibited, it is convenient to classify mechanisms for cell activation into several general categories:
a) Positive feedback mechanisms: There exists a class of inflammatory reactions that are mediated by direct action of plasma inflammatory stimulators (oxygen free radicals, platelet activating factor (PAF), cytokines (e.g., TNF-a, IL-1, IL-8), complement fragments, endotoxins, coagulation and fibrinolytic factors, leukotrienes, thrombin, and oxidized LDL). The list of inflammatory mediators is long, and may in part be triggered by trauma or by bacterial, viral, or fungal sources.
b) Negative feedback mechanisms: An alternative pathway for cell upregulation in the microcirculation is by depletion of anti-inflammatory factors. This list is shorter and includes nitric oxide, adenosine, glucocorticoids, and selected cytokines (e.g., IL-10).
c) Contact activation: A specialized form of cell activation by membrane contact has been proposed in the form of juxtacrine activation. A non-activated endothelial cell may be stimulated during membrane contact by an activated leukocyte and vice versa, e.g., by oxygen free radical production in the membrane contact region between the cells and by formation of platelet activating factor (PAF) and other bioactive lipids.
d) Activation by mechanotransduction: Alternative forms of cell activation due to either a shift to un-physiologically low or high fluid shear stresses acting on the endothelium or a shift in the oxygen supply to the tissue. Fluid shear serves as a control mechanism for various forms of cell activation and the expression of anti-inflammatory and proinflammatory genes. Inflammatory stimulators of the sort listed above can influence the fluid shear response via a cGMP-mediated mechanism.
e) Activation by physical transients: Transients of gas (like oxygen, carbon dioxide, etc.) concentrations or temperature transients have the ability to stimulate cell activation irrespective of the direction of the transient (up or down) but dependent on the magnitude of the transient.
   Over the lifetime of an individual, it is likely that several, if not all, of these mechanisms may at one time or another stimulate inflammation. The challenge is to identify prevailing mechanisms. What is needed is a hypothesis for hypertension and the metabolic X-syndrome, in which inflammation, as part of daily nutrition and interaction with the environment, causes long-term cell activation and inflammation.

Within the field of nutrition, the mechanisms associated with an attenuated response to insulin administration in Type II Diabetes remain uncertain. Equally uncertain are the mechanisms that often accompany Type II Diabetes and Metabolic Syndrome X, such as immune suppression, high blood pressure and inflammation which in turn lead to microvascular complications and eventually to tissue failure.

In diabetes, the brush border barrier has been shown to be compromised and leaky to digestive enzymes (“leaky gut syndrome”), precipitating auto-digestion. In addition to damage to the intestine, auto-digestion causes at least two other complications:
a) Breakdown products of a body's own proteins and lipids are generated in the intestinal wall, and released into the circulation where they generate further inflammation, and
b) Digestive enzymes leak into the blood stream where they reach all other organs and continue to break down the body's own proteins, lipids and other tissue components.
   These combined actions lead to inflammation as seen in diabetes. In addition, leakage of digestive enzymes (e.g., serine proteases) into the circulation activates other pro-enzymes (e.g., pro-metalloproteinases) and in the process amplifies tissue degradation. It may be that this process plays a critical role in the complications that accompany diabetes.

In the blood plasma of a Type II diabetic or an individual with Metabolic Syndrome X experimental model, the digestive enzymes reach high levels of activity. Many proteins are therefore subject to potential cleavage and loss of function. One especially important aspect is the cleavage of the extracellular domain of receptors involved in basic physiological functions. For example, it has been proven (using antibody binding against the extracellular domain receptors) that active plasma proteases cleave the extracellular domain of the insulin receptor and leukocyte adhesion molecules, causing insulin resistance and immune suppression, respectively. With every receptor type that is cleaved the function of the cell is further compromised. Receptor cleavage has been observed in an animal model with hypertension, immune suppression and with insulin resistance.

Arterial hypertension, as seen in diabetic patients and individuals with Metabolic Syndrome X, is associated with a risk for inflammation and greatly accelerated organ dysfunction. Evidence from different experimental models of hypertension indicates a multitude of microvascular complications that span across all segments of the microcirculation with both high and low blood pressures. The origin of this organ injury is still incompletely understood.

In addition to signaling activity and a possible cause for an elevated arterial blood pressure, oxygen free radicals have been proposed as potential sources for organ injury. An interesting feature of hypertensives is that a variety of complications occur besides the elevated blood pressure, such as immune suppression with impaired leukocyte endothelial adhesion and CD18 downregulation or insulin resistance. These complications have not been conclusively linked to oxygen free radical production. Other mechanisms may be involved in organ injury. Few investigations exist that have probed into potential proteolytic activity as a root cause of the aforementioned symptoms. The deleterious effects of receptor cleavage have not been properly addressed nor has any successful therapeutic measure been used to prevent the inflammation and impaired cellular functioning observed in patients with Hypertension, Type II Diabetes, Metabolic Syndrome X, or the like.

Thus, there is a need in the healthcare industry for an effective method of treating inflammation and organ dysfunction caused by high blood pressure, improper membrane receptor cleavage, auto-digestion, and/or oxygen free radical production. The technique should be simple to use, efficient, accurate and able to produce predictable and effective results in a relatively short period of time.

### SUMMARY OF THE INVENTION

The present invention relates to the use of matrix-degrading metalloproteinase inhibitors to counteract a specific cascade involved in tissue inflammation and organ damage as observed in diseases including but not limited to Hypertension, Type II Diabetes, and Metabolic Syndrome X. Part of the basis of the present invention stems from past observations and a recent discovery which has now shed new light on a mechanism for hypertension. The evidence indicates that hypertensives suffer from multiple defects, including a defect in vascular leukocyte adhesion, shear stress response and insulin transport, in addition to a defect in arteriolar tone with superoxide overproduction and inadequate nitric oxide formation. It has been shown that the plasma of hypertensives exhibits enhanced protease activity. This evidence suggests that membrane receptors that mediate these conditions may be cleaved by the proteases and protease inhibition will prevent this effect. Additionally, the origins of organ injury and inflammation resulting from hypertension and similarly related diseases (e.g., Type II Diabetes and Metabolic Syndrome X) are not completely understood. Without such knowledge, it is difficult to effectively treat the diseases since the root cause of the symptoms cannot be targeted. In fact, only until now has the inflammatory cascade involved in these diseases been revealed by the present invention. The present invention has identified matrix-degrading metalloproteinases (MMPs) as key players in inflammation and organ damage. Using such knowledge, the present invention proposes the use of an MMP inhibitor to treat and/or prevent tissue inflammation and organ damage by normalizing the blood plasma's protease activity, reducing blood pressure, preventing membrane receptor cleavage, and reducing the levels of oxygen free radicals in the blood stream

In one exemplary embodiment, the present invention is a compound for treating inflammatory pathologies and syndrome. The compound includes an effective dose of an MMP inhibitor, resulting in decreased inflammatory symptoms.

In another exemplary embodiment, the present invention is a compound to treat inflammatory pathologies and syndrome. The compound includes an effective dose of an MMP inhibitor, resulting in lowered blood pressure and lowered pancreatic proteinase activity.

In yet another exemplary embodiment, the present invention is a method of treating inflammation. The method includes providing an effective dose of an MMP inhibitor to treat inflammation, resulting in lowered hypertension.

In another exemplary embodiment, the present invention is a method of treating syndrome x. The method includes providing an effective dose of an MMP inhibitor to treat metabolic syndrome x, resulting in decreased pancreatic proteinase activity.

In another exemplary embodiment, the present invention is a method for treating disease. The method includes exposing abnormally functioning cells to a substance that decreases the amount of MMPs present within their cytoplasm.

In another exemplary embodiment, the present invention is a method of treating disease. The method includes exposing cells to a substance that is capable of decreasing blood plasma protease activity, reducing oxygen free radical formation, and inhibiting cleavage of membrane receptors.

In yet another exemplary embodiment, the present invention is a method for treating disease. The method includes introducing about 5.4 mg/kg/day of an MMP inhibitor daily into a patient's circulatory system for a defined period of time.

### BRIEF DESCRIPTION OF THE DRAWINGS

FIG. 1A shows digital images of MMP-9 levels, as detected by labeling with primary antibody and Vector NovaRED substrate in select circulating leukocytes of SHR and WKY before and after treatment with an MMP inhibitor, doxycycline, according to the present invention.

FIG. 1B shows a comparison of MMP-9 distribution between SHR and WKY on individual neutrophils as determined by optical density measurements.

FIG. 2A shows selected low magnification micrographs of microvessels and interstitium of WKY and SHR mesentery after MMP-9 immunolabeling with Vector NovaRED substrate.

FIG. 2B shows selected micrographs at higher magnification of rat microvessels and interstitium in WKY and SHR mesentery after immunolabeling for MMP-9.

FIG. 2C shows a comparison of MMP-9 protein levels as detected by light absorption measurement of Vector NovaRED substrate in mesentery of control WKY and SHR with and with doxycycline treatment.

FIG. 3A shows digital fluorescent micrographs of WKY and SHR mesenteric microvessels labeled with fluorogenic peptide substrate showing matrix metalloproteinase (MMP-2,9) enzymatic activity.

FIG. 3B shows a comparison of MMP-9 activity levels as detected by fluorescent substrate intensity in mesentery of control WKY and SHR without and with doxycycline treatment.

FIG. 4 shows plasma protease activity values as determined by fluorescent units without (control) and with doxycycline treatment.

FIG. 5A shows bright field micrographs of rat mesentery after TNBT labeling in WKY and SHR before and after doxycycline treatment.

FIG. 5B shows light absorption measurements of zymogen deposits in the rat mesentery of SHR and WKY rats without and with treatment by doxycycline.

FIG. 6A shows typical micrographs of immunolabel (Vector NovaRED) for the extracellular domain of the insulin receptor α on fresh leukocytes (neutrophils and monocytes) from WKY and SHR.

FIG. 6B shows insulin receptor α density measured by light absorption after labeling with a primary antibody against the extracellular domain of the receptor and Vector NovaRED substrate.

FIG. 7A shows typical micrographs of fresh leukocytes (neutrophils and monocytes from a Wistar rat strain) on a blood smear after labeling with the extracellular domain of CD18 integrin and after exposure to WKY and SHR plasma with and without chronic treatment of doxycycline.

FIG. 7B shows normalized light intensity of CD18 label on leukocytes exposed to the plasmas of WKY and SHR with and without chronic treatment of doxycycline relative to the values on a naive Wistar donor with a value of 1).

FIG. 8A shows selected immunohistochemical micrographs of SHR leukocytes before (SHR) and after chronic doxycycline treatment (SHR Doxycycline) with Nova Vector RED substrate labels of primary anti-body against NFκ-B (Left panels).

FIG. 8B shows that NFκ-B is present in both cytoplasm and in the cellular nuclei of control SHR, an effect that is significantly reduced by chronic MMP inhibition.

FIG. 9 shows selected bright field (A), fluorescent (B), and merged (C, with pseudocolor) images of frozen sections of rat thymus after TUNEL labeling (in panels A) and propidium iodide (PI) labeling (in panels B). PI-positive nuclei (B) are used to identify the total number of cells in the fields.

### DETAILED DESCRIPTION OF THE INVENTION

The present invention is based in part on a series of studies that uncovered a unique and previously unrecognized mechanism of inflammatory mediator production involving the powerful pancreatic enzymes in the intestine. These enzymes, fully activated and concentrated in the lumen of the intestine, are the key component of normal digestion. They are highly concentrated and optimized to digest almost all biological tissues, including proteins, lipids, nucleotides and carbohydrates, of both mammalian and amphibian source. Digestion of the gut itself is largely prevented by compartmentalization of the activated pancreatic enzymes within the lumen of the intestine by the mucosal epithelial (brush border) cells. The limited permeability of the mucosal layer under ideal physiological conditions prevents the high molecular weight digestive enzymes from escaping into the wall of the intestine.

But under a variety of conditions, including diabetes, hypertension, and aging, this mucosal barrier may not always be as tight, and digestive enzymes may escape into the interstitial space of the wall of the intestine and begin to digest the intestinal wall. In the course of this process, a largely undescribed class of powerful inflammatory mediators (derived entirely from digestion of extracellular and cellular components by pancreatic enzymes) is released. We refer to these as the inflammatory fragments generated by digestive enzymes. These inflammatory fragments escape into the portal venous circulation, pass into the central circulation and have the ability to reach all organs to generate an inflammatory cascade. They also escape into the intestinal lymphatics as well as directly into the peritoneum and thereby bypass the normal hepatic detoxification. We refer to this sequence of events as the self-digestion process.

Such particular pathway described above involves the pancreatic enzymes in the intestine which lead to MMP activation in general, e.g., in acute shock. The present inventors also have preliminary results indicating elevated levels of pancreatic enzymes in the plasma of chronic experimental hypertensives. Such finding would suggest that another pathway may exist which functions by over-expression of MMP in the microcirculation and in the tissue parenchyma which in turn is due to the enhanced oxygen free radical formation in hypertensives and animals with metabolic X syndrome. The enhanced oxygen free radical formation leads to translocation of selected nuclear promoters that control expression of pro-inflammatory and anti-inflammatory genes and MMP expression. MMP expression in genetic hypertensives may be an event that may occur with and even without an involvement of the intestine. Further studies will shed more light on the details of the mechanism. Thus, in the present application, the case of genetic hypertensives are also included, in which MMP expression and activity may be produced also locally in the tissue due to a free radical formation which in turn causes proinflammatory gene expression and anti-inflammatory gene suppression in which the MMP expression is part of the genes involved.

A basis for the present invention is the hypothesis that inflammation in hypertension, diabetes, and aging is due to self-digestion with cleavage of receptors and other vascular proteins. Pancreatic digestive enzymes, especially serine proteases and lipases, are a major source for production of humoral inflammatory mediators that escape in small amounts and trigger inflammation in the microcirculation. Furthermore, new evidence suggests that the normal diet of purpose-bred rats contains inflammatory mediators obtained after digestion with pancreatic enzymes, so that the choice of the diet may be an important determinant for the degree of leakage of pancreatic digestive enzymes across the epithelium of the mucosal barrier. There may also exist amplification mechanisms for self-digestion facilitated by escape of digestive proteases into the circulation and into the extracellular matrix, which cause activation of zymogen forms of matrix metalloproteinases (MMP) in remote organs and further enhancement of self-digestion, and cause insulin resistance, peripheral cardiovascular complications, and progressive tissue degeneration.

The present invention is based in part on the examination of the development of enzymatic activity and inflammatory markers in the presence of chronic treatment with oral serine protease and matrix metalloproteinase (MMP) inhibitors. What is considered is the protease/lipase transport across the mucosal barrier and its activity in the wall of the intestine, in plasma, and in selected vital organs by zymography. These values are correlated with glucose kinetics, inflammatory markers (in-vivo leukocyte activation, oxygen free radical production, c-reactive proteins, cytokines), and aging indices. Examples and data are presented with in vivo dynamics within the mesentery for sake of simplicity. Such results are applicable to other tissue systems as well.

The present invention provides for treatment with a broad acting MMP inhibitor to block the recently discovered elevation of enzymatic activity in experimental forms of arterial hypertension. Such treatment leads to normalization of the SHR arterial blood pressure, blockade of the elevated levels of oxygen free radical production in the microcirculation, normalization of the free elevated levels of MMP 2, 9 (and other proteolytic and lypolytic enzyme) activity, normalization of immune cell counts, reduced apoptosis and normalization of the insulin resistance to levels of asymptomatic wild type rat strains. The treatment also serves to reduce similar parameters slightly elevated in the Wistar Kyoto Rat (WKY) rat strain, a rat strain that also has mild form of hypertension.

At its core, the present invention is based on the finding that the inhibition of MMPs mitigates the symptoms of diseases rooted in inflammation: Metabolic Syndrome X, Hypertension, Diabetes, etc. Thus, by using MMP inhibitors, the present invention proposes novel methods for treatment of such inflammation-based diseases. In the examples used herein and throughout this disclosure, doxycycline is used as an exemplary MMP inhibitor for sake of simplicity. However, the present invention is not limited to doxycycline or other synthetic or natural MMP inhibitors. Any MMP inhibitor, or other similar product, that serves to control, decrease or prevent plasma protease activity in diabetics and hypertensives, would also be within the scope of the present invention, and would assist in the controlling of inflammation in the diseases presented herein. Additionally, in particular embodiments and examples presented herein, such methods are described with respect to treatment of Hypertension, Type II Diabetes, and Metabolic Syndrome X, but it should be noted that such disease applications are not limiting and the present invention may be utilized to treat any class of diseases which act via an inflammatory cascade involving or including MMPs.

Further, the application of doxycycline or other MMP inhibitors, to patients or populations, may be in either conventional dosed form or in a novel mode described herein, namely, nutriceutical application. Such form of providing an MMP inhibitor to a population would entail adding a natural MMP inhibitor to the diet (e.g., into drinking water), to enable the mitigation of inflammatory symptoms. Natural MMP inhibitors include, but are not limited to, grape seed extract, cranberry fruit extract, pomegranate fruit extract, and green tea extract, among others. Such MMP inhibitors may be added to all drinking (tap) water, in certain bottled waters or juices, or in specialty drinks or other foods. The purpose would be to deliver MMP inhibitors, such as doxycycline or others, to a population in a proper dosage such that the benefits of its anti-inflammatory properties are gained without causing an excess in MMP inhibitor intake.

In order to show an example of the mechanism as described in this disclosure and the effects of a protease inhibitor on such mechanism, experiments have been performed on the spontaneously hypertensive rat (SHR). It has been shown that in the SHR, a model of essential hypertension in man, a previously undescribed mechanism exists that leads to organ injury and cardiovascular complications due to metalloproteinase activation (MMP 2, 9, and probably others). A group of SHR and its control strain, the Wistar Kyoto (WKY) rat, were treated for a period of 10-12 weeks with a broad acting MMP inhibitor (doxycyline, 55 mg/Liter) in drinking water.

Reference will now be made in detail to the various experiments leading to the present invention. It is to be understood that the following detailed description is provided to further explain details of various embodiments of the invention, and is not provided as an exhaustive description of all substances that can be used according to the invention, or all steps that can be performed in practicing the methods of the invention. Rather, it is provided to describe certain details of embodiments of the invention, which will provide those of ordinary skill in the art a more thorough understanding of various embodiments, which can be applied to other embodiments of the aspects of the invention, without requiring those artisans to practice undue experimentation to achieve the full scope of the claimed invention.

The experiments as delineated herein offer substantial evidence that the inflammatory cascade as observed in the aforementioned diseases may be effectively blocked using an MMP inhibitor thereby reducing blood pressure, decreasing proteolytic activity in the plasma, preventing improper receptor cleavage, and reducing oxygen free radical formation. The methods used to ascertain the significance of the present invention and the results obtained from experimental tests are set forth below along with a discussion of the data and the pertinent conclusions drawn. All references made via head notes are provided near the end of this disclosure in an effort to more clearly describe the experiments and steps leading up to the present invention.

Animals—For the experiments described with respect to the present invention, studies were carried out with mature male Spontaneous Hypertensive Rats, or SHR (300-400 gm, 14-16 weeks) and their normotensive controls, the Wistar Kyoto (WKY) of comparable age and eight under general anesthesia (Nembutal, 50 mg/kg I.M.). Selected WKY and SHR were treated with doxycycline (55 mg/liter in drinking water, average consumption ˜5.4 mg/kg/day, West-Ward Pharmaceutical Corp., Eatontown, N.J.) for a period of 24 weeks. It should be noted that lower and higher concentrations are also effective. A range of effective does of about 1.0-20.0 mg/kg/day, resulting from the dissolution of about 5.5-220 mg of an MMP inhibitor per liter, may also be used to produce an effective result. Narrower ranges of MMP solute may also be used, for example, 75-180 mg, and 100 mg-150 mg.

Blood Cells—Fresh leukocytes were fixed on a blood smear in whole blood (using heparin as anticoagulant, 10 U/ml) with formalin solution and labeled with a primary antibody (10%, neutral buffered, 1 hr). Total leukocyte count was determined with a hemocytometer, and hematocrit was centrifuged in microhematocrit tubes. Neutrophils were separated in a density gradient (Histopaque-1077, Sigma Aldrich, spun at 42g for 5 min).

Mesentery Microcirculation—The ileocecal portion of the mesentery was prepared as described in Delano and Schmid-Schonbein, “The Glucocorticoid and Mineralocorticoid Receptor Distribution in a Microvascular Network of the Spontaneously Hypertensive Rat.” Briefly, arterial blood pressure was recorded via a femoral artery catheter. Individual sectors of mesentery were exposed via an abdominal midline incision while superfused with Krebs-Henseleit buffer (37° C., pH=7.4).

Immuno-histochemical Labeling of MMP—Freshly exposed mesentery was fixed by superfusion with formalin solution (10%, neutral buffered, 1 hr). The tissue was excised and postfixed in the same formalin solution for 24 hours to permit full penetration of the primary antibody and the sample was then processed for in situ labeling. The time period from initial anesthesia to fixation of the mesentery was kept below 60 minutes to minimize de novo syntheses of MMPs during the experiment.

The cellular distribution of matrix metalloproteinase proteins (MMP-9, gelatinase-B) in whole mount mesentery specimens was delineated by a biotin/avidin immunolabeling technique (Vectastain Elite ABC Kit, Vector Laboratories, Inc.). A peroxidase enzyme substrate (Vector NovaRED, Vector Laboratories, Inc.) was used to visualize MMP-9 on tissue specimens after primary antibody labeling (Santa Cruz Biotechnology). Tissue specimens without primary antibody against MMP-9 served as control and they showed no detectable labeling in line with previous experiments. No counterstain was applied to facilitate quantitative labeling intensity measurements. All microvascular structures could be readily identified on the labeled specimen. The immunolabeling procedures were carried out under standardized conditions to permit quantitative comparison of the MMP levels among mesentery tissue specimens.

Plasma Protease Activity—Fresh plasma samples were frozen (−60 C) until measurements were taken. On the day of the experiment, the samples were unfrozen and tested simultaneously for overall protease activity with fluorescent protease assay kit (Enzchek, Cat #E-6638, Molecular Probes). The substrate was cleaved by a range of proteases (metallo-, serine, acid and sulfhydryl proteases). Protease activity was determined from fluorescent intensity after peptide cleavage (in fluorescent units).

In-vivo Zymography—Measurement of MMP-1,9 activity was obtained by superfusion of the mesentery with fluorogenic substrate (0.5 μmol/L; catalogue number D2293, Sigma-Aldrich Inc., St. Louis, Mo.) added to the suffusate. The substrate was cleaved by MMP-1 (collagenase-1) and MMP-9 (gelatinase B). The mesentery was loaded with substrate 10 minutes prior to image collection and continuously suffused with substrate throughout the experiment. The associated fluorescence was visualized by epi-illumination at 280 nm passing through a 340 nm emission filter on a fluorescent intravital microscope (Leica), and recorded digitally for measurement of fluorescent intensity. Light intensity of the emitted fluorescent light was recorded in digital units (1-256) after subtraction of background intensity in the absence of the tissue with standardized microscope settings. Reproducibility of these measurements as determined by repeated measurements on the same specimen was within 3%.

TNBT Labeling for detection of superoxide formation in mesentery microcirculation—The production of oxygen free radical formation in the mesentery microcirculation was evaluated by reduction of nitroblue tetrazolium to formazan, a reaction that can be blocked with superoxide dismutase as previously shown in DeLano et al., “Control of Oxidative Stress in Microcirculation of Spontaneously Hypertensive Rat” and in Kobayashi et al., “Oxidative Stress Promotes Endothelial Cell Apoptosis and Loss of Microvessels in the Spontaneously Hypertensive Rats.” Briefly, fresh tetranitroblue tetrazolium (TNBT, Glucose Oxidase Substrate Kit II, Vector Laboratories; Burlingame, Calif.) (prepared about every 10 min from a fresh solution) was topically applied by constant drip for 1 hr on selected mesentery sectors. At the end of this period, Krebs-Henseleit (37° C., 7.4 pH) was used to wash TNBT from the specimen for 15 min. The mesentery tissue was then fixed with formalin solution (10%, neutral buffered, Sigma Diagnostics, St. Louis, Mo.; topical application) for 15 min, excised, and stored in formalin (10%).

Images of the tissue were generated by digital bright field microscopy and the formazan levels were measured in the form of light absorption, as described above for measurements of the Vector NovaRed substrate density.

Insulin Receptor and CD18 Labeling—To examine the possibility that proteases in plasma of the SHR may cleave the extracellular domain of surface receptors, fresh Wistar leukocytes were exposed for 1 hr to plasma from SHR, WKY and control Wistar rats (0.1 ml leukocyte suspension:0.5 ml plasma 100%, 37° C.). The cells were then spread on a blood smear, fixed (10% formalin, neutral buffered) and labeled with a primary antibody against the extracellular domain of the insulin receptor (Rα, N-20, sc-710 polyclonal antibody mapping to the N-terminus, Santa Cruz Biotech) followed by the biotin/avidin labeling technique as described above. To label CD18, an antibody was used against the extracellular domain (epitope mapping at the N-terminus of β2 integrin, Santa Cruz Biotechnology, CA) and the same labeling technique.

Digital Image Analysis—Images of the immunolabel density were recorded at different magnifications, from relatively low power overviews of the tissue (10× objective, 10× objective) to higher magnification of single cells (at 100× oil immersion objective, numerical aperture 1.40). The images were recorded digitally under standard light conditions and fixed settings of the substage condenser.

Single images were recorded with a digital camera (FujiFilm FinePix S1 Pro, Fuji Photo Film Co., Ltd., Tokyo, Japan) and continuous video records with color charge coupled device camera (DEI470, Optronics Engineering, Goleta, Calif.) at fixed light settings, so that the camera served as a quantitative light intensity meter. Images were digitized and analyzed on a laboratory computer to minimize operator error (NIH Image, 1.61, public domain software, spatial resolution of 640×480 pixels).

The density of the immune substrate label (e.g. Vector NovaRed) was measured on selected segments in the microcirculation in form of a light absorption (A), such that A=In (I/Iₒ). I is the light intensity over the tissue and Iₒ is the incident light intensity without the tissue.

The light intensities on the images were determined in the following fashion unless indicated otherwise. Single leukocytes were analyzed by placement of a digital window on the image of the cell surface such that light intensities were determined as average values over the cell cytoplasm. In the case of microvascular images, a narrow optical window of thickness ˜2 μm and length ˜30 μm was placed over the endothelial cell and light intensity was determined as the average over the endothelium. This approach was kept unchanged over the course of the study.

Statistics—The light intensity and light absorption measurements of MMP-9 were grouped by rat strain and are presented as mean±standard deviation. Unpaired comparisons of mean values between animal groups were carried out by the Student's t-test and p<0.05 was considered significant.

Blood Pressures and Heart Rate—The blood pressure of WKY and SHR was reduced by doxycycline treatment as shown below in Table 1, a trend that was detectable in terms of systolic, diastolic or mean blood pressure. After doxycycline treatment, the elevated blood pressure of the SHR reached values comparable to the WKY before treatment, while the arterial pressure of WKY rats was reduced to values below the blood pressure of the Wistar rat (about 105 mmHg) from which the WKY and SHR strains were bred. The heart rates were not significantly affected by the MMP inhibition.

TABLE 1
WKY and SHR Central Hemodynamics

| | |
|---|---|
| Control Groups | Doxycycline Groups |

| | WKY (n = 3) | SHR (n = 3) | WKY (n = 6) | SHR (n = 7) |
|---|---|---|---|---|
| Mean Blood Pressure | 134.4 ± 10.1* | 174.2 ± 11.7 | 95.7 ± 8.5** | 126.3 ± 13.5** |
| (mmHg) | | | | |
| Systolic Blood Pressure | 168.2 ± 15.2* | 216.3 ± 14.1 | 125.3 ± 10.6** | 161.9 ± 14.5** |
| (mmHg) | | | | |
| Diastolic Blood Pressure | 110.5 ± 8.7* | 141.9 ± 11.3 | 77.4 ± 6.8** | 99.4 ± 17.3** |
| (mmHg) | | | | |
| Heart Rate (beats/min) | 349 ± 25* | 401 ± 30 | 364 ± 30** | 398 ± 60** |

*P < 0.05 WKY Control vs SHR Control. WKY Doxycycline vs SHR Doxycycline
**P < 0.05 WKY Control vs WKY Doxycycline. SHR Control vs SHR Doxycycline
n = number of animals.

Blood Cell Counts—The hematocrit of WKY and SHR were substantially the same and slightly decreased after doxycycline treatment as shown in Table 2. In contrast the leukocyte counts of both WKY and SHR fell during the treatment, with the SHR group falling to values less than 50% of its usually high count in the circulation.

TABLE 2
WKY and SHR Hematocrit and Leukocyte Count

| | Control Groups | | Doxycycline Groups | |
|---|---|---|---|---|
| | WKY (n = 3) | SHR (n = 3) | WKY (n = 6) | SHR (n = 7) |
| Hematocrit | 39.8 ± 1 | 40.8 ± 0.4 | 37.2 ± 0.8*, ** | 36.8 ± 0.8** |
| Leukocyte Count | 3770 ± 247* | 6330 ± 747 | 2737 ± 272*, ** | 2532 ± 574** |

*P < 0.05 WKY Control vs SHR Control, WKY Doxycycline vs SHR Doxycycline
** P < 0.05 WKY Control vs WKY Doxycycline. SHR Control vs SHR Doxycycline
n = number of animals.

Plasma Protease Activity—In another experiment, and in order to explore the utility of chronic protease inhibition, a group of mature normotensive Wistar Kyoto rats (as well as a cohort of spontaneously hypertensive rats) were treated with a broad acting metalloproteases inhibitor (doxycycline, 55 mg/liter in drinking water, average consumption ˜5.4 mg/kg/day) over a period of 12 weeks until the age of 30 weeks. Such treatment serves to significantly reduce the central blood pressure elevation observed over time in these animals (Table 3), dramatically reduce the plasma protease activity (Table 4) and the MMP 2,9 tissue activity in the mesentery microcirculation, and reduce the superoxide production (by nitroblue tetrazolium detection, NBT) as well as the cleavage of the leukocyte adhesion integrin Mac1 (CD18). These results indicate that the enzymatic activity in plasma has the ability to cleave important membrane receptors including the insulin receptor, the amino-acid transporters for L-arginine to facilitate NO production and many others.

TABLE 3
Mean Arterial Blood Pressure (mmHg)
during Chronic MMP Inhibition

[symbol]

| | | Spontaneously |
|---|---|---|
| Groups | Wistar Kyoto rat | Hypertensive rat |
| Control Rats | 134.4 ± 10.1 (3 | 174.2 ± 11.7 (3 |
| | rats) | rats)* |
| After MMP | 95.7 ± 8.5 (5 rats) | 126.3 ± 13.5 (7 |
| inhibition (for 12 | | rats)* |
| weeks) | | |

*p < 0.01 vs untreated control [symbol]
Age = 30 weeks at time of measurement

TABLE 4
Plasma Protease Activity (FU)
During Chronic MMP Inhibition

[symbol]

| | | Spontaneously |
|---|---|---|
| Groups | Wistar Kyoto rat | Hypertensive rat |
| Control Rats | 1764 ± 656 (3 rats) | 3289 ± 342 (3 |
| | | rats)* |
| After MMP | 413 ± 40 (4 rats) | 472 ± 24 (7 rats)* |
| inhibition (for 12 | | |
| weeks) | | |

*p < 0.03 vs untreated control [symbol]
Age = 30 weeks at time of measurement

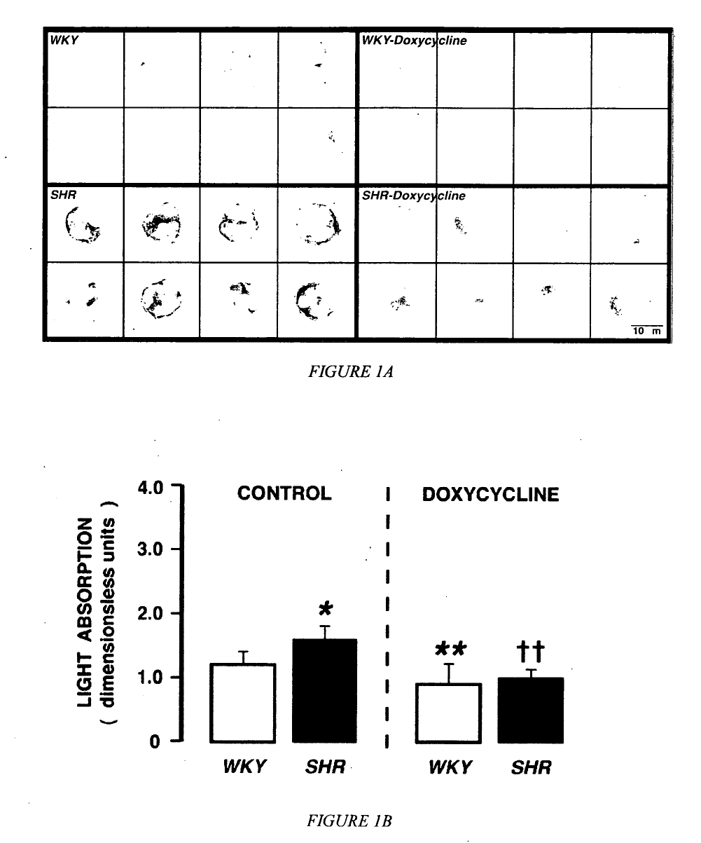

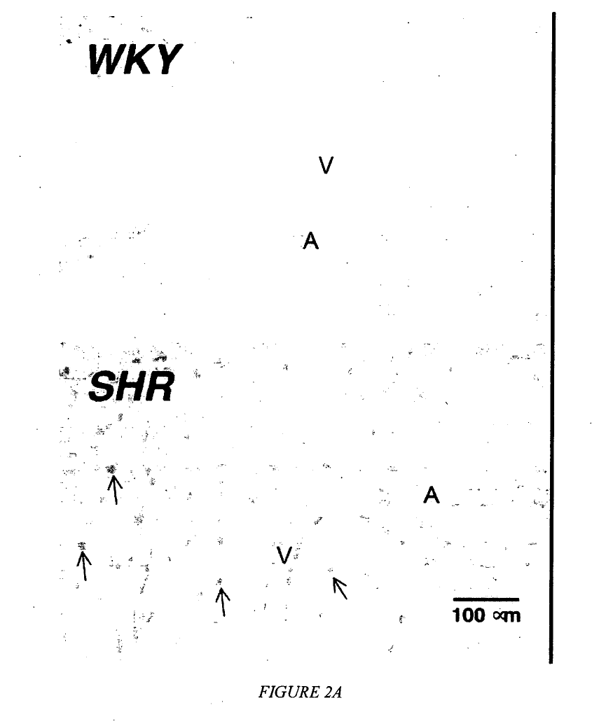

Matrix Metalloproteinase (MMP-9) in Neutrophils—Experiments showed that the anti-MMP-9 antibody label was located predominantly in the neutrophil cytoplasm as shown in FIG. 1A. In FIG. 1A, the digital images of MMP-9 levels, as detected by labeling with primary antibody and Vector NovaRED substrate in select circulating leukocytes of SHR and WKY before (left panels) and after treatment (right panels) with doxycycline, are shown. Undetectable levels of substrate labeling were observed in the absence of the primary antibody (results not shown). The MMP-9 distribution on individual neutrophils as determined by optical density measurements showed on average significantly higher values in the SHR as shown in FIG. 1B thereby confirming the visualization in FIG. 1A that doxycycline is an effective inhibitor of MMP-9 found in the WKY and SHR. The number of measurements in each group in FIG. 1B is 90 cells derived from 3 animals in each animal group. *p<0.05 versus WKY, **p<0.05 versus WKY without doxycycline treatment (control), †† p<0.05 versus SHR without doxycycline. Examination of the labeling pattern among leukocytes suggests that both WKY and SHR have cells with low levels of MMP-9, but the SHRs have more cells with stronger labels. After doxycycline treatment, the MMP-9 levels fell to average values without significant differences between strains as shown in FIG. 1A and FIG. 1B.

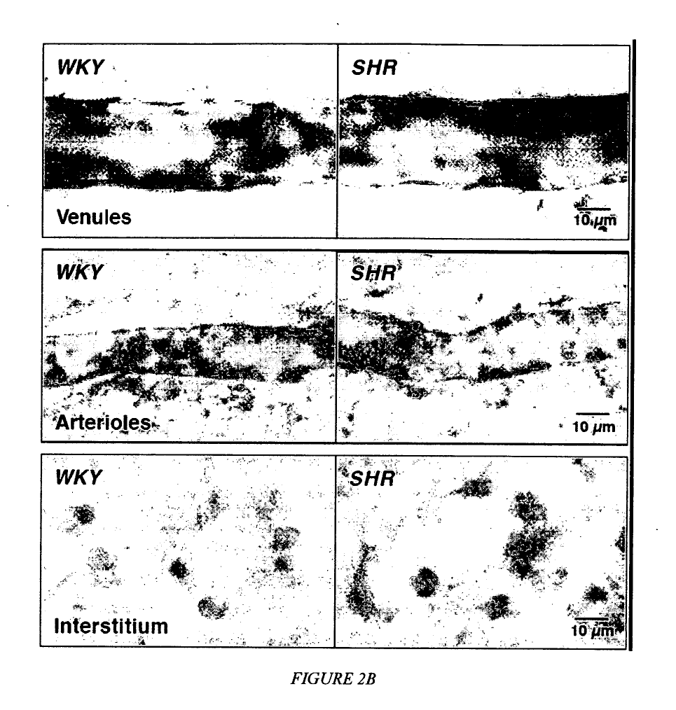

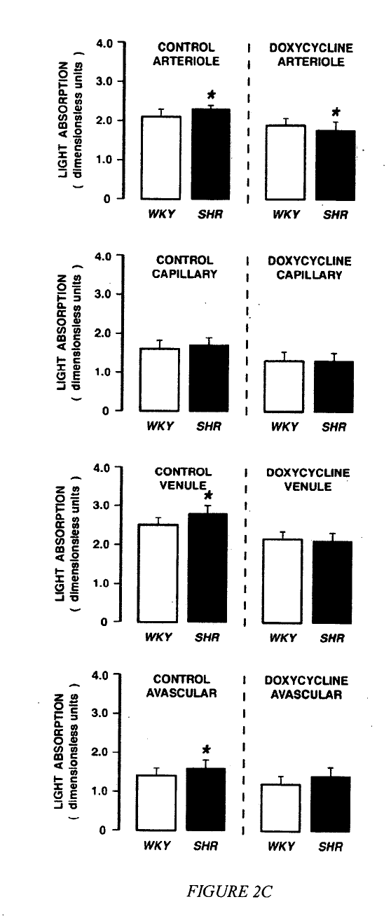

MMP-9 Protein Levels in Mesentery Microcirculation—Undetectable levels of Vector NovaRed label are present in mesentery specimens without primary antibody or in specimens treated with a non-binding antibody (not shown). MMP-9 protein is detectable in all major structures of the mesentery including the endothelium and interstitial cells. The general pattern of the MMP-9 protein labeling in arterioles, capillaries and venules is similar for both WKY and SHR mesentery and can be observed in FIG. 2A. FIG. 2A shows selected low magnification micrographs of microvessels and interstitium of WKY and SHR mesentery after MMP-9 immunolabeling with Vector NovaRED substrate. From the figure, the pronounced labeling in SHR endothelial cells of arterioles (A) and venules (V) as well as in interstitial mast cells (arrows) is observed. Endothelial cells exhibit a non-uniform pattern of protein labeling such that cells with enhanced protein label are located side by side with cells that have low or even undetectable levels of substrate label. Venules for both WKY and SHR revealed an increased MMP-9 receptor density when compared with comparable arterioles.

The majority of mast cells have enhanced levels of MMP-9 compared with fibroblast or mesothelial cells in the mesentery as shown in FIG. 2B. FIG. 2B shows selected micrographs at higher magnification of rat microvessels and interstitium in WKY and SHR mesentery after immunolabeling for MMP-9. Lower levels of the substrate label are present in arteriolar smooth muscle cells and no red blood cells were significantly labeled. The average antibody labeling density as determined by light absorption measurements over randomly selected arterioles and venules in the mesentery is significantly higher in the SHR as shown in FIG. 2C. FIG. 2C therefore shows MMP-9 protein levels as detected by light absorption measurement of Vector NovaRED substrate in mesentery of control WKY and SHR without and with doxycycline treatment. The number of measurements in each microvessel type and in the avascular area is 90 derived from 3 rats in each animal group. *p<0.05 versus WKY. No difference in the level of MMP-9 protein levels of capillaries was detected between WKY and SHR. Avascular tissue areas of the mesentery sheet in SHR showed a significantly enhanced level of MMP-9.

After doxycycline treatment the MMP 9 levels decreased significantly in both WKY and SHR as shown in FIG. 2C. The SHR reached control levels that were not significantly different from the values in the WKY rats.

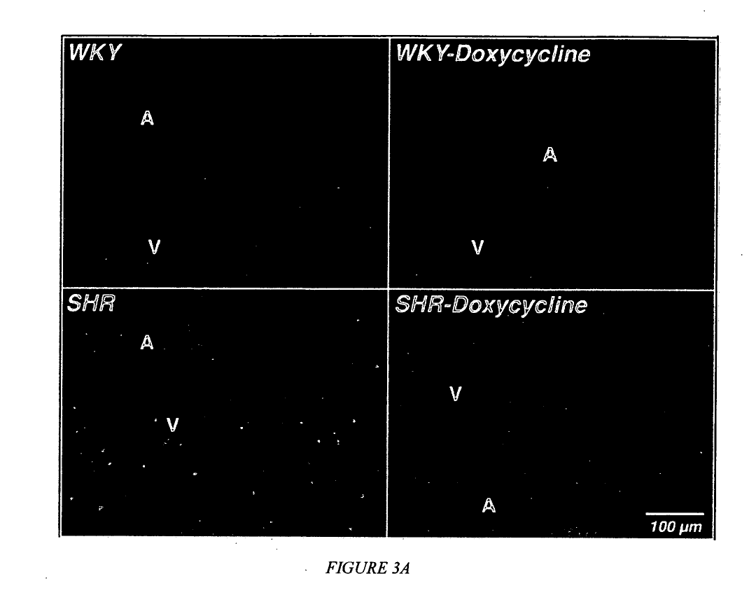

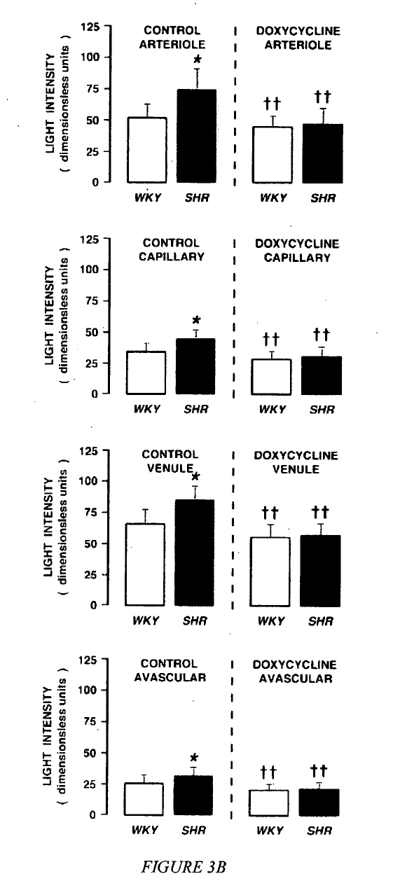

MMP-9 Activity in Mesentery Microcirculation—MMP-1,9 enzymatic activity as detected by fluorogenic substrate cleavage is present in all major structures of the mesentery including the endothelium and interstitial cells as shown in FIG. 3A. FIG. 3A shows digital fluorescent micrographs of WKY and SHR mesenteric microvessels labeled with fluorogenic peptide substrate showing matrix metalloproteinase (MMP-2,9) enzymatic activity. Arterioles (A) and venules (V) are visible. From FIG. 3A, it is noted that there is an enhanced fluorescent emission over the endothelial cells and mast cells in the SHR, an effect that is less detectable after the doxycycline treatment.

The microvascular distribution of the MMP-1 and MMP-9 activity closely resembles the pattern observed by MMP-9 immunolabeling which was shown in FIG. 2A. Endothelial cells have a non-uniform pattern of substrate cleavage such that cells with enhanced enzyme activity may be located side by side with cells with low or even undetectable levels of substrate label. Venules for both WKY and SHR revealed an increased MMP-9 receptor density when compared with comparable arterioles. The majority of mast cells exhibited enhanced levels of MMP-9 enzyme activity compared with fibroblast or mesothelial cells in the mesentery. The average peptide substrate density as determined by digital light intensity measurements over randomly selected mesenteric microvessels (arterioles, capillaries, venules) is significantly higher in the SHR for the MMP-9 enzyme as shown in FIG. 3B. This figure shows MMP-9 activity levels as detected by a fluorescent substrate intensity in mesentery of control WKY and SHR without and with doxycycline treatment. The number of measurements in each vessel type and in the vascular area is 90 derived from 3 mesenteries in each animal group. *p<0.05 versus WKY, ††p<0.05 versus same strain without doxycycline treatment. Avascular tissue areas of the mesentery sheet in SHR showed a significantly enhanced matrix metalloproteinase (MMP-9) enzyme activity. Lower levels of the fluorogenic substrate label were observed in smooth muscle cells and no red blood cells showed measurable fluorescent activity. Doxycycline treatment led to a significant reduction of the MMP activity in endothelial cells and fibroblasts in both rat strains as shown by FIG. 3A and FIG. 3B. The intensities in the SHR reached levels that were the same as in the control WKY rats. Application of the metal chelator ethylenediaminetetraacetic acid (EDTA, 5 mM) reduced the fluorescent intensity generated by the fluorogenic substrate to such low values that were undetectable with the current imaging system (results not shown). This observation supports the notion that MMPs are one of the major enzymes responsible for substrate cleavage.

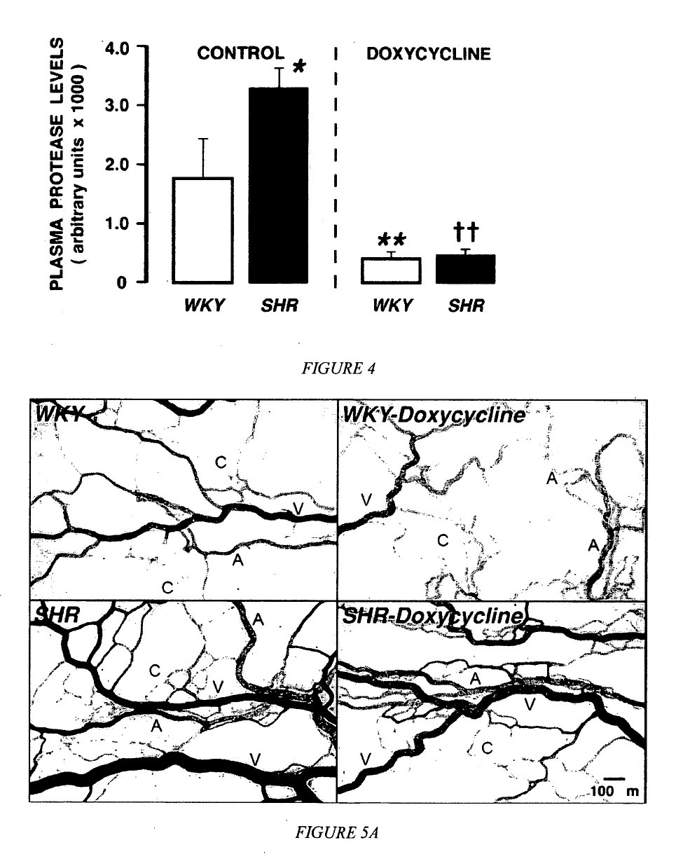

Plasma Protease Activity—The SHR has a significantly elevated plasma protease activity shown in FIG. 4 as detected by the fluorogenic substrate that is dramatically reduced by the doxycycline treatment. FIG. 4 shows plasma protease activity values as determined by fluorescent units without (control) and with doxycycline treatment. The number of measurements is n=3 in control WKY and SHR and n=4 in the doxycycline treated WKY and SHR cells. *p<0.05 versus WKY, **p<0.05 versus WKY without doxycycline treatment, and ††p<0.05 versus SHR without doxycycline. The plasma protease activity in the WKY rats is also reduced in animals subject to the chronic MMP inhibition.

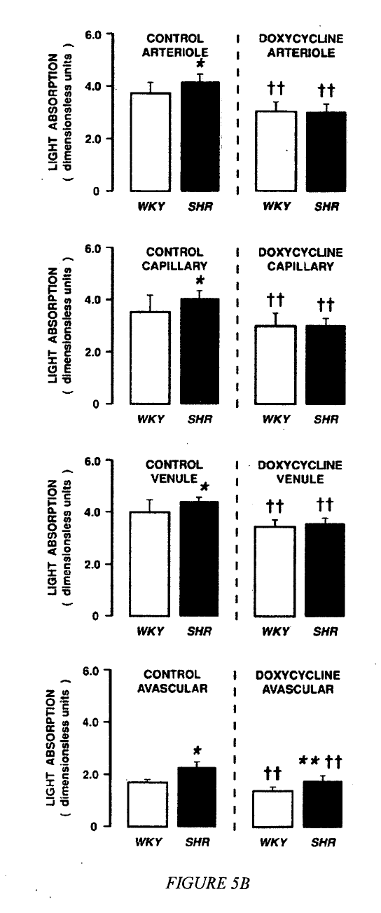

Oxygen Free Radical Formation In Rat Mesentery Microcirculation—The elevated free radical production in the mesentery microcirculation of the SHR, as detected by NBT labeling, is reduced by blockade of MMPs as shown in FIG. 5A. FIG. 5A shows bright field micrographs of rat mesentery after TNBT labeling in WKY and SHR before and after doxycycline treatment. There appears to be a reduction of dark (blue/red) tetrazolium deposits in all microvessel types, arterioles (A), capillaries (C), and venules (V), after doxycycline treatment. This reduction is observed not only in the arterioles but even more notable in the low-pressure venules and in the interstitial cells. Doxycycline treatment also served to reduce the zymogen deposits in the normotensive WKY rats, a feature that is confirmed by the quantitative light absorption measurements as shown in FIG. 5B. Thus, FIG. 5B shows light absorption measurements of zymogen deposits in the rat mesentery of SHR and WKY rats without and with treatment by doxycycline. Mean±standard deviation in each blood vessel type and in the avascular area is 30 per rat with 3 rats in each animal group. *p<0.05 versus WKY, ††p<0.05 versus same rat strain without doxycycline treatment, **p<0.05 versus WKY with doxycycline treatment.

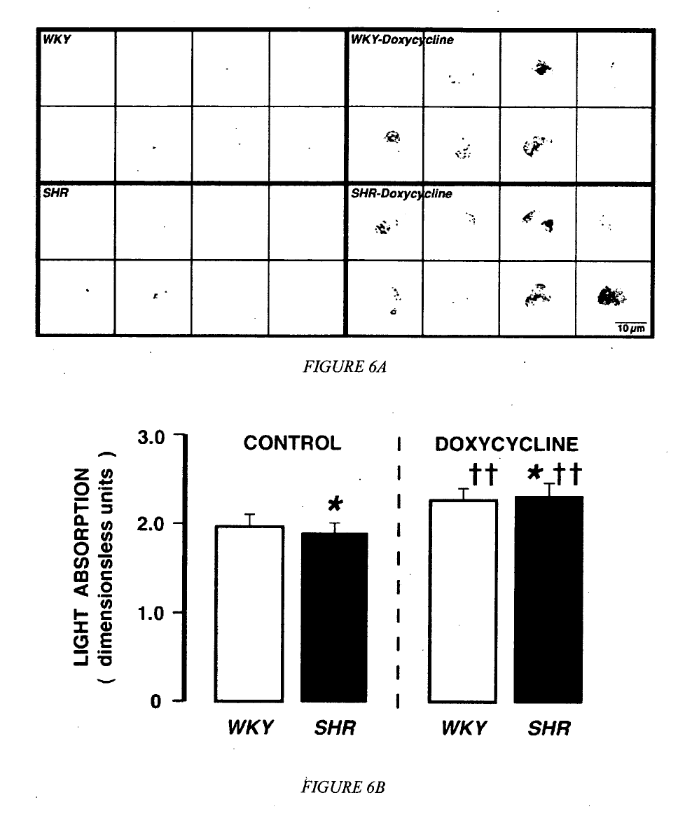

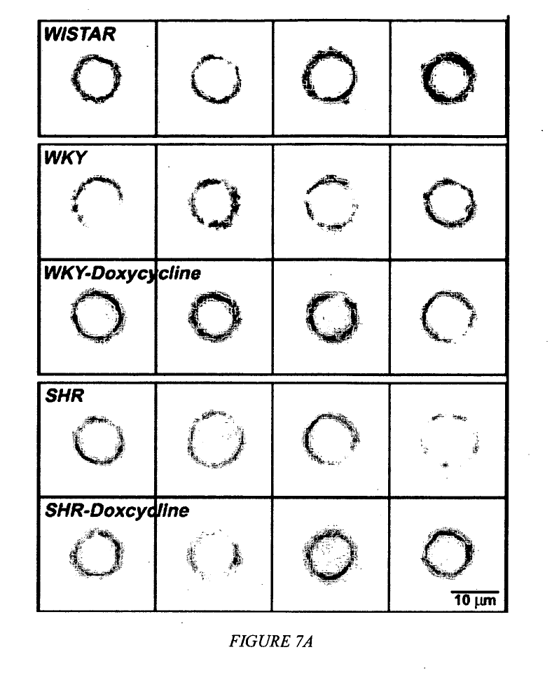

Receptor Cleavage in the SHR—Immunolabeling of the extracellular domain of the insulin receptor α on fresh leukocytes shows reduced levels in the SHR as shown in FIG. 6A. FIG. 6A shows typical micrographs of immunolabel (Vector NovaRED) for the extracellular domain of the insulin receptor α on fresh leukocytes (neutrophils and monocytes) from WKY and SHR. The left panels show leukocytes from control rats and the right panels from rats after doxycycline treatment. There is considerable cleavage of the receptor in the WKY rats. The levels of insulin receptor label on leukocytes are significantly enhanced after doxycycline treatment in both WKY and SHR as shown in FIG. 6B. Thus, FIG. 6B shows insulin receptor α density measured by light absorption after labeling with a primary antibody against the extracellular domain of the receptor and Vector NovaRED substrate. Groups are the same as those shown in FIG. 6A. Mean±standard deviation in each group is derived from 30 cells per rat with 3 rats in each animal group. *p<0.05 versus WKY, †\p<0.05 versus same strain without doxycycline treatment.

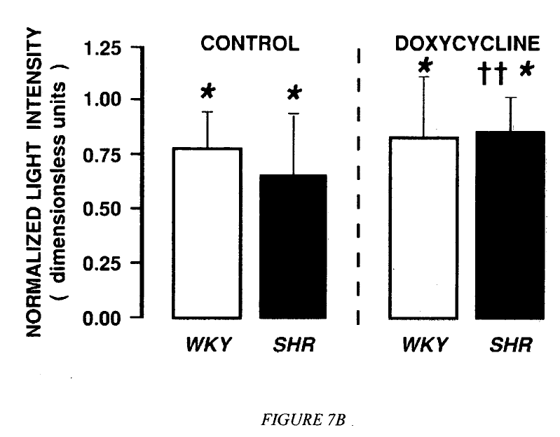

Exposure of naive leukocytes from normotensive Wistar rats to plasma from WKY or SHR causes on average a significant reduction of the extracellular domain of CD18 by about 25 to 35% as shown in FIG. 7A and FIG. 7B. FIG. 7A shows typical micrographs of fresh leukocytes (neutrophils and monocytes from a Wistar rat strain, top row) on a blood smear after labeling with the extracellular domain of CD18 integrin. The Wistar cells were exposed for 30 minutes to plasma from WKY and SHR without (control) and with chronic doxycycline treatment. The results shown in FIG. 7A indicate that plasma from the SHR and also the WKY rat has the ability to cleave the extracellular domain of membrane receptors to a degree that exceeds the activity of their normotensive Wistar control. In FIG. 7B, the normalized light intensity of CD18 label on leukocytes exposed to the plasmas of WKY and SHR with and without chronic treatment of doxycycline relative to the values on a naive Wistar donor (with a value of 1) is shown. The intensity measurements were made over a ring region of individual leukocytes with insulin receptor label. Mean±standard deviation in each group is derived from 30 cells per rat with 3 rats in each group. *p<0.05 versus values in Wistar rat leukocytes, ††p<0.05 versus same strain without doxycycline treatment. In light of FIG. 7B, there appears to be support for the notion that doxycycline normalizes the amount of CD18 integrins in the SHR.

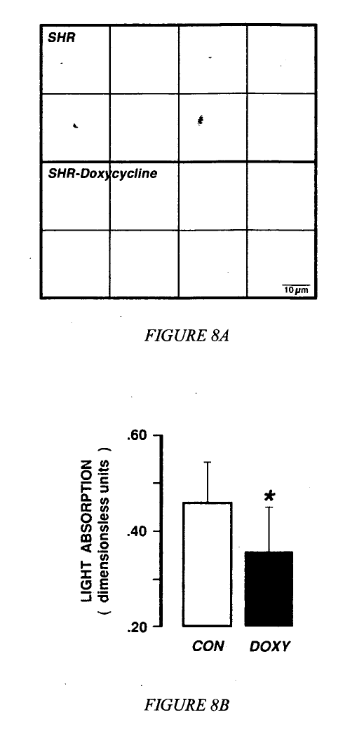

Additional experiments were performed to study the effects of SHR organ injury after MMP blockade, which supported the position that MMP inhibition attenuates organ injury mechanisms in the SHR. In this study, the Nuclear Factor NFκ-B expression in the spontaneously hypertensive rat (SHR) was considered. An immunohistochemical analysis of NFκ-B expression in SHR leukocytes (neutrophils and monocytes) was performed before and after MMP blockade (with 24 week doxycycline treatment). The measurements were carried out in fresh leukocytes, to facilitate analysis of NFκ-B density in the nucleus without overlapping cells. The NFκ-B was labeled under standardized conditions with a mAb against p50 (sc-7178, Santa Cruz Biochemicals), and the NFκ-B label density was measured over the nucleus (see FIG. 8A). The measurements were carried out by digital optical density measurements.

FIG. 8A shows selected immunohistochemical micrographs of SHR leukocytes before (SHR) and after chronic doxycycline treatment (SHR Doxycycline) with Nova Vector RED substrate labels of primary anti-body against NFκ-B (Left panels). Note that NFκ-B is present in both cytoplasm and in the cellular nuclei of control SHR, an effect that is significantly reduced by chronic MMP inhibition, as shown in FIG. 8B (*, †p<0.05 versus control (CON) by Student t-test). The observations are confirmed by quantitative light absorption measurements in the nuclear domains of the cell cytoplasm (right panels). Absorption values are derived from 90 randomly selected cells of three rats in each group and 3 separate measurements per cell in cell cytoplasm and nucleus. Note the nucleus was not counterstained to facilitate optical density measurements since it can be readily identified by light diffraction on leukocytes spread out into pancake shapes on a blood smear.

The analysis shows that NFκ-B is distributed in SHR leukocytes throughout the cell cytoplasm and with translocation into the nucleus. Chronic MMP inhibition (Doxycyline) serves to strongly attenuate the NFκ-B translocation in the SHR. This observation confirms that the SHR is subject to an inflammatory cascade with enhanced expression of genes that are under the control of NFκ-B

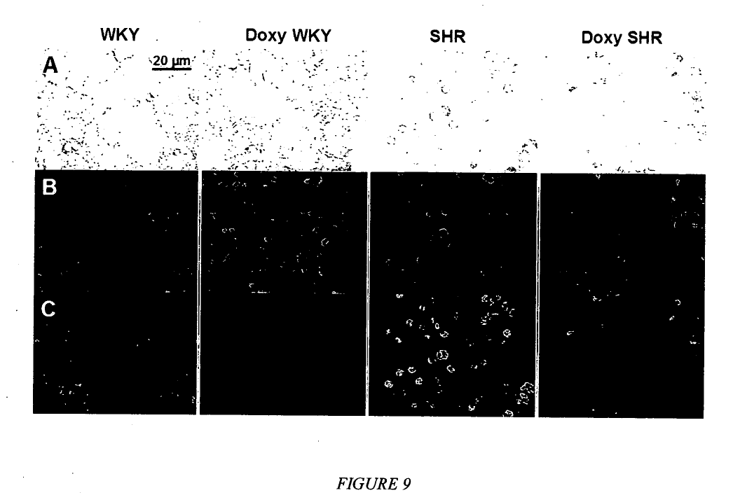

A subsequent study was performed to consider the reduction of cell apoptosis after chronic MMP inhibition. In addition to the dramatic reduction of blood pressure, free radical production, membrane receptor cleavage and other pathophysiological parameters (described elsewhere) that point towards organ injury in the SHR, cell apoptosis was also considered after MMP inhibition. The analysis was carried out in the thymus, which has previously been shown to exhibit loss of organ weight and lymphocyte apoptosis in the SHR (Suzuki et al., Enhanced DNA fragmentation in the thymus of spontaneously hypertensive rats, Am. J. Physiol., 276:H2135-H2140, 1999). For this purpose, a novel digital technique was introduced to determine nuclear fragmentation on freshly-harvested frozen tissue sections labeled for DNA breaks with the TUNEL technique and with propidium iodide (P1) for the detection of cell nuclei (see FIG. 9).

FIG. 9 shows selected bright field (A), fluorescent (B), and merged (C, with pseudocolor) images of frozen sections (about 7 μm thickness) of rat thymus after TUNEL labeling (in panels A) and propidium iodide (PI) labeling (in panels B). PI-positive nuclei (B) are used to identify the total number of cells in the fields. Their image is superimposed on the bright field image of TUNEL-positive nuclei (dark brown color in A) to determine digitally the light absorption due to the TUNEL label (shown in green in panels C) and the level of apoptosis (C). The enhancement of cell apoptosis in the SHR thymus is noted, which is greatly attenuated by chronic MMP blockade with doxycycline treatment (see Table 5).

Table 5 shows that the fraction of apoptotic cells in the SHR thymus is reduced by chronic MMP inhibition to a level equal to apoptosis in normotensive controls (the Wistar Kyoto rat, WKY). Apoptosis, as detected by the TUNEL technique, is also reduced in the SHR skeletal muscle (spinotrapezius muscle) but not in kidney. The reduced apoptosis in the thymus by MMP blockade is also confirmed by direct measurements of the organ weight (shown in Table 6). There are no significant reductions of thymus apoptosis in the WKY rats after chronic MMP blockade. These results further show that chronic MMP inhibition serves to attenuate cell and organ injury in the spontaneously hypertensive rat.

TABLE 5
Fraction Of Apoptotic Cells In Different Tissues (%)

| | WKY | Doxy WKY | SHR | Doxy SHR |
|---|---|---|---|---|
| Thymus | 6.1 ± 4.0 | 4.7 ± 3.0 | 43.0 ± 20.6* | 13.8 ± 9.1^{††} |
| Kidney | 5.1 ± 5.1 | 5.1 ± 3.5 | 10.4 ± 4.3 | 12.8 ± 5.2 |
| Skeletal | 0.8 ± 1.1 | 1.4 ± 1.3 | 3.2 ± 2.6 | 1.7 ± 2.4^{†} |
| muscle | | | | |

Values are means ± SD. Each group has 10 samples from 2 animals.
Measurements were carried out in a tissue area of 10,000 μm².
*P < 0.05 P < 0.01 compared with WKY.
^{†}P < 0.05 or ^{††}P < 0.01 compared with the untreated group.

TABLE 6
Thymus Weight Normalized With Respect To Body Weight

| | WKY | Doxy WKY | | Doxy SHR |
|---|---|---|---|---|
| | (n = 3) | (n = 5) | SHR (n = 3) | (n = 5) |
| Thymus wt, | 78.8 ± 5.1 | 81.4 ± 7.4 | 72.3 ± 7.5 | 87.4 ± 3.3^{††} |
| mg/100 g body | | | | |
| wt | | | | |

Values are means ± SD;
n, number of animals.
^{††}P < 0.01 compared with sham-operated SHR.

The results presented in the various figures described herein show that the SHR has elevated protease activities in plasma and in microvascular endothelium that may cause cleavage of key membrane receptors. Chronic blockade of the protease activity with a broad acting MMP inhibitor reduces blood pressure and normalizes plasma protease activity, reduces MMP protein and oxidative stress levels in microvessels) normalizes blood cell count and reduces receptor cleavage. Protease activity may constitute a fundamental defect encountered in the SHR that is associated with its insulin resistance and suppression of leukocyte adhesion to the endothelium.

Microvascular display of MMP activity—The studies demonstrate the utility of a fluorescent indicator in conjunction with intravital microscopy and immuno-histochemistry to delineate protease activity in microvessels as an important inflammatory activity. The use of a fluorogenic substrate for detection of matrix metalloproteinases (MMP-1,9) provides an intravital microscopic display of activity that is an important aspect of the pathophysiology. The fact that the metal chelating agent EDTA suppresses the light emitted after cleavage of the fluorescent substrate confirms the central role of MMPs in the proteolytic activity of the SHR.

Immunolabeling of the intact microcirculation indicates that there are enhanced levels of MMP-9 in microvascular endothelium in a pattern that closely follows the protease activity detected with a substrate for MMP-1,9 as shown in FIG. 2A and FIG. 3A. The restriction to microvessels and their endothelium is also in line with the pattern of NBT reduction by oxygen free radicals as shown in FIG. 5A. Both oxygen free radical production and MMP expression appear to be present in microvascular endothelium, which is in line with the observation that either superoxide scavenging or inhibition of MMP activity may alleviate a variety of symptoms in the SHR.

It is also noted that MMP-9 protease was present in tissue fibroblast as shown in FIG. 2A, which is also co-localized with the protease activity as shown in FIG. 3A. Studies show that mechanical stress on vascular smooth muscle cells enhances message for MMP-1 and proenzyme release via an NADPH oxidase pathway, an enzyme which is also enhanced in the wall of the same mesentery microvessels in which the MMP activity is observed in the current studies. Enhanced oxidative stress can trigger the nuclear transcription factor NFκB as signal transducer, which in turn leads to MMP expression. But the present studies consistently showed these events in the SHR not only on the arterial side of the microcirculation but also prominently on the venous side and even in circulating leukocytes that periodically pass through both high and low pressure regions of the circulation. The lack of a unique arteriolar localization of markers for organ injury is observed also with markers for apoptosis or other oxidative enzymes. Thus, there is a possibility that the MMPs in the SHR may be induced through a mechanism other than just elevated blood pressure possibly involving oxygen free radicals. In human hypertensives the plasma MMP activity has been correlated with enhanced systolic blood pressure and arterial stiffening.

Pathophysiological aspects of MMP activity in SHR—MMPs exert diverse pathophysiological effects ranging from proteolytic remodeling of extracellular matrix in various physiologic situations (developmental tissue morphogenesis, tissue repair, angiogenesis) to pathogenic roles in excessive breakdown of connective tissue components. MMP activity has been observed in many of the complications that accompany a hypertensive state. For example, in the left ventricle with age there is a marked increase in MMP-1 mRNA while at the same time a decrease in the WKY rat. Activation of MMP leads to decreased cardiac tissue tensile strength and may cause systolic and diastolic dysfunction. Left ventricular volume and MMP-2 activity in obese male spontaneously hypertensive heart failure rats are enhanced. After cerebral ischemia, SHRs were reported to have an acute increase in type IV collagenase (MMP-9) that is maintained over several days compared with control brain while gelatinase A (MMP-2) is elevated only after several days.

Furthermore, in situ zymography of brain slices in stroke prone SHR show an increase in plasminogen activator/plasmin activity that co-localizes with the cerebral damage. Concomitant MMP-2 activation is only observed in damaged brain area. Thus, urokinase-type plasminogen activator is expressed and selectively catalyses proteolysis in the injured area of spontaneous brain damage in stroke prone SHR.

A mechanism that may induce MMP activity in the SHR—Application of elevated blood pressure and stretch of endothelial cells leads to MMP expression. In smooth muscle cells the stretch-dependent MMP-2 expression and release depends on the p47phox subunit of NADPH oxidase. However, several pieces of evidence suggest that the elevated MMP protein levels are the consequence of another event. The high expression of MMP is prominent not only in arterioles but also in venules with a blood pressure that is indistinguishable between WKY and SHR and even in interstitial cells that are surrounded by fluid pressure close to zero (relative to atmospheric pressure). The close co-localization observed at the level of the microcirculation between the MMP protein levels and the TNBT reduction as shown in FIG. 3 and FIG. 6 supports the possibility that the MMP expression may be associated with the oxidative stress in the same endothelium. The elevated oxidative stress in the SHR versus WKY endothelium is also present in SHR endothelial cultures (unpublished results) under identical fluid stresses, in line with the hypothesis that this oxidative stress is of genetic origin. Enhanced levels of superoxide, hydrogen peroxide and peroxynitrite formation modulate and activate MMP-2 and 9 activity or inactivate TIMPs. There is a link between oxygen free radical formation, NFκ-B activation and translocation to the nucleus and MMP expression. An elevated level of pancreatic elastase level, as observed in the SHR aorta, may also play a role in the conversion of pro-MMP to active MMP. Endothelin-1 may also modulate MMP-2 synthesis and activation in aging SHRs.

In DOCA-salt Hypertension, endothelin A receptors regulate cardiac MMP activity, vascular remodeling and fibrosis. The MMP system appears to be early activated before the development of Hypertension and is possibly mediated by endothelin-1. MMP-9 levels are readily induced by embolic and mechanical ischemia/reperfusion in the brain and closely associated with ischemic injury.

MMP inhibition—Besides its antimicrobial activity, doxycycline inhibits a variety of MMPs, including MMP 1, 2, 8 and 9, and blocks serpinolytic activity (degradation of α-1-antitrypsin) and plasmin. It is in the class of natural MMP inhibitors analogs with a high affinity for bivalent ion. It also inhibits in endothelial cells the induction of MMP 8 and 9 or in epithelial cells induction of MMP-9 after TGF stimulation via a MAPK pathway.

Besides inhibition of MMP-1 activity doxycycline also inhibits collagen synthesis in vitro and has the potential to block angiogenesis. While it is evident that in the current situation doxycycline likely blocked MMP-1 and 9, other actions on the SHR remain to be explored.

Although the above examples were presented with doxycycline as an exemplary MMP inhibitor, the present invention is not limited to doxycycline, but may be any MMP inhibitor, whether they are in the same strucutural family as doxycycline or not. Other examples of MMP inhibitors which may be used and are thus within the scope and purview of the present invention include, but are not limited, to, Mmonocylin, tetracyclin, batimastat (BB-94, BB-2983), quinapril (an ACE inhibitor), PD166793, PD200126, PD166793, PG-530742, Marimastat, and others either known to one having ordinary skill in the art or currently under development. It may be that any compound that is capable of blocking the Zn binding site of an MMP to block its catalytic activity may be useful and used for the present invention.

A more generalized structure that may be useful for the present invention may be, for example, any compound containing hydroxamic acids that chelates the catalytic Zn++ in MMPs. There are also new compounds in development that target the Zn binding sites which may use different chemistry.

The mode of delivery of the MMP inhibitor to a patient or population may be numerous and other modes, not specifically described above, are within the scope of the present invention and within the purview of one having ordinary skill in the art. For example, a pill, a capsule, and IV under acute conditions may be used to normalize a severely hypertensive patient.

Plasma protease activity—The present results described above were designed to determine whether protease activity could be determined in the plasma of the SHR. The specific protease(s) involved may include trypsin, chymotrypsin, elastase as well as amylases, such as heparinase, and lipases.

Receptor cleavage—The current results provide evidence for proteolytic activity in SHR plasma and on endothelial cells. Enzyme activity is also observed to some degree in the low blood pressure WKY, a strain that has elevated arterial pressure compared to the normal Wistar strain. The enzymatic activity may be associated with receptor cleavage. Membrane CD18, whose expression levels are reduced on neutrophils in the SHR, is cleaved by its plasma as shown in the present study in FIG. 7. Blockade of the MMP activity served to attenuate this abnormality.

The SHR also has reduced P-selectin expression levels on postcapillary venules, a situation that leads to a reduced rolling interaction between leukocytes and the endothelium on these postcapillary venules. The presence of elevated soluble P-selectin levels in hypertensives is in line with such receptor cleavage.

Furthermore, it is noted in the SHR cleavage of the insulin receptor that may be mediated by enzymatic activity in its plasma. The loss of insulin receptor sites may be associated with insulin resistance in the SHR. The insulin receptor cleavage is blocked by MMP inhibition. The receptor cleavage may be in line with the observation that glycation of proteins in genetic (Type II) and also streptozotocin-induced (Type I) diabetic rats is reduced by doxycycline and by non-antimicrobial chemically modified tetracycline derivatives.

Thus, protease activity in hypertension may not only affect suspended proteins but also lead to cleavage of membrane receptors. This unchecked enzymatic activity may be one of the main reasons for the diverse number of dysfunctions encountered in the SHR.

The above discussion shows that SHR has enhanced MMP-9 protein levels and MMP-1 and MMP-9 activity in the microcirculation in addition to enhanced plasma protease levels. MMP inhibition with doxycycline leads to reduction of blood pressure and oxygen free radical formation and cleavage of the extracellular domain of CD18 and the insulin receptor. The results point the first time to a mechanism that may explain diverse cell dysfunctions encountered in hypertensive, such as insulin resistance or immune suppression. Further studies based on the results presented herein on protease activity will determine what other receptor activity may be compromised by enzymatic activity. Knowledge to this effect may lead to useful clinical interventions against the cell dysfunction and organ injury mechanisms that accompany hypertension. Receptor cleavage may also be the reason for diverse abnormalities encountered in patients with hypertensive or metabolic X syndrome, and needs further investigation.

It is expected that similar treatment in other forms of diseases with Metabolic Syndrome X (diabetics, other forms of arterial, venous and pulmonary hypertension), will attenuate the inflammatory reaction in the circulation and thereby provide a significant clinical benefit against organ injury and organ failure. Such treatment could reduce the level of insulin resistance, enhance the fluid shear stress response, reduce blood pressure, reduce the tendency for blood clotting and thrombosis, enhance leukocyte adhesion to the endothelium and thereby restore the specific immune response and the acute repair to injury, and reduce apoptosis in hypertensives and diabetics and similar syndromes in aging.

Potential uses of the present invention are many. It may serve as alternative treatment for control of inflammation in essential hypertensive patients, patients with the metabolic syndrome x, in patients with aging hypertension, as well as type 1 and 2 diabetics. It includes improvement of insulin response, reduced thrombosis, reduced apoptosis and normalization of arterial dilation and immune response.

The following references were either cited directly or were used to present some of the observations found with respect to MMP activity. They are therefore incorporated by reference herein in their entirety into this disclosure for further support and explanation on how the results and conclusions were obtained for the present invention. A list of all cited references and other relevant sources is provided as well to provide adequate background and support for the present invention and its conclusions: (1) Aoki T, Sumii T. Mori T, Wang X, and Lo E H. Blood-brain barrier disruption and matrix metalloproteinase-9 expression during reperfusion injury: mechanical versus embolic focal ischemia in spontaneously hypertensive rats. Stroke 33: 2711-2717, 2002; (2) Arndt H, Smith C W, and Granger D N. Leukocyte-endothelial cell adhesion in spontaneously hypertensive and normotensive rats. Hypertension 21: 667-673, 1993; (3) Asanuma K, Magid R. Johnson C. Nerem R M, and Galis Z S. Uniaxial strain upregulates matrix-degrading enzymes produced by human vascular smooth muscle cells. Am J Physiol Heart Circ Physiol 284: H1778-1784, 2003; (4) Bursztyn M, Ben-Ishay D, and Gutman A. Insulin resistance in spontaneously hypertensive rats but not in deoxycorticosterone-salt or renal vascular Hypertension. Journal of Hypertension 10: 137-142, 1992; (5) Cakir Y and Hahn K A. Direct action by doxycycline against canine osteosarcoma cell proliferation and collagenase (MMP-1) activity in vitro. In Vivo 13: 327-331, 1999; (6) Camp T M, Smiley L M, Hayden M R, and Tyagi S C. Mechanism of matrix accumulation and glomerulosclerosis in spontaneously hypertensive rats. J Hypertens 21: 1719-1727, 2003; (7) DeLano F A, Balete R, and Schmid-Schonbein G W. Control of oxidative stress in microcirculation of spontaneously hypertensive rats. Am J Physiol Heart Circ Physiol 288: H805-812, 2005; (8) Delano F A, Parks D A, Ruedi J M, Babior B M, and Schmid-Schönbein G W. Microvascular Display of Xanthine Oxidase and NADPH Oxidase in the Spontaneously Hypertensive Rat. Microcirculation in review, 2006; (9) Delano F A and Schmid-Schonbein G W. Microvascular Display of Xanthine Oxidase and NADPH Oxidase in the Spontaneously Hypertensive Rat. Microcirculation in press, 2006; (10) Delano'F A and Schmid-Schönbein G W. The glucocorticoid and mineralocorticoid receptor distribution in a microvascular network of the spontaneously hypertensive rat. Microcirculation 11: 69-78, 2004; (11) DeLano F A and Schmid-Schönbein G W. A possible role of matrix metalloproteinases in cellular injury of the spontaneously hypertensive rat. FASEB J 17: A346.330, 2003; (12) DeLano F A and Schmid-Schönbein G W. Visualization of enhanced matrix metalloproteinase activity in the spontaneously hypertensive rat by a fluorogenic substrate. FASEB J 19: A1263, 2005; (13) Duivenvoorden W C, Hirte H W, and Singh G. Use of tetracycline as an inhibitor of matrix metalloproteinase activity secreted by human bone-metastasizing cancer cells. Invasion Metastasis 17: 312-322, 1997; (14) Ergul A, Portik-Dobos V, Giulumian A D, Molero M M, and Fuchs L C. Stress upregulates arterial matrix metalloproteinase expression and activity via endothelin A receptor activation. Am J Physiol Heart Circ Physiol 285: H2225-2232, 2003; (15) Frears E R, Zhang Z, Blake D R, O'Connell J P, and Winyard P G. Inactivation of tissue inhibitor of metalloproteinase-1 by peroxynitrite. FEBS Lett 381: 21-24,1996; (16) Galis Z S and Khatri J J. Matrix metalloproteinases in vascular remodeling and atherogenesis: the good, the bad, and the ugly. Circ Res 90: 251-262, 2002; (17) Griffin M O, Jinno M, Miles L A, and Villarreal F J. Reduction of myocardial infarct size by doxycycline: a role for plasmin inhibition. Mol Cell Biochem 270: 1-11, 2005; (18) Grote K, Flach I, Luchtefeld M, Akin E, Holland S M, Drexler H, and Schieffer B. Mechanical stretch enhances mRNA expression and proenzyme release of matrix metalloproteinase-2 (MMP-2) via NAD(P)H oxidase-derived reactive oxygen species. Circ Res 92: e80-86, 2003; (19) Hanemaaijer R, Sorsa T. Konttinen Y T, Ding Y, Sutinen M, Visser H, van Hinsbergh V W, Helaakoski T. Kainulainen T, Ronka H, Tschesche H, and Salo T. Matrix metalloproteinase-8 is expressed in rheumatoid synovial fibroblasts and endothelial cells. Regulation by tumor necrosis factor-alpha and doxycycline. J Biol Chem 272: 31504-31509, 1997; (20) Hanemaaijer R, Visser H, Koolwijk P, Sorsa T, Salo T, Golub L M, and van Hinsbergh V W. Inhibition of MMP synthesis by doxycycline and chemically modified tetracyclines (CMTs) in human endothelial cells. Adv Dent Res 12: 114-118, 1998; (21) Hulman S, Falkner B, and Freyvogel N. Insulin resistance in the conscious spontaneously hypertensive rat: euglycemic hyperinsulinemic clamp study. Metabolism: Clinical and Experimental 42: 14-18, 1993; (22) Kim H S, Luo L, Pflugfelder S C, and Li D Q. Doxycycline inhibits TGF-beta1-induced MMP-9 via Smad and MAPK pathways in human corneal epithelial cells. Invest Ophthalmol Vis Sci 46: 840-848, 2005; (23) Kobayashi N, DeLano F A, and Schmid-Schonbein G W. Oxidative stress promotes endothelial cell apoptosis and loss of microvessels in the spontaneously hypertensive rats. Arterioscler Thromb Vasc Biol 25: 2114-2121, 2005; (24) Kolev K, Skopal J, Simon L, Csonka E, Machovich R, and Nagy Z. Matrix metalloproteinase-9 expression in post-hypoxic human brain capillary endothelial cells: H₂O₂ as a trigger and NF-kappaB as a signal transducer. Thromb Haemost 90: 528-537, 2003; (25) Kuzuya M and Iguchi A. Role of matrix metalloproteinases in vascular remodeling. J Atheroscler Thromb 10: 275-282, 2003; (26) Lamparter S, Slight S H, and Weber K T. Doxycycline and tissue repair in rats. J Lab Clin Med 139: 295-302, 2002; (27) Lehoux S, Lemarie C A, Esposito B, Lijnen H R, and Tedgui A. Pressure-induced matrix metalloproteinase-9 contributes to early hypertensive remodeling. Circulation 109: 1041-1047, 2004; (28) Lenda D M, Sauls B A, and Boegehold M A. Reactive oxygen species may contribute to reduced endothelium-dependent dilation in rats fed high salt. Am J Physiol Heart Circ Physiol 279: H7-H14, 2000; (29) Li H, Simon H, Bocan T M, and Peterson J T. MMP/TIMP expression in spontaneously hypertensive heart failure rats: the effect of ACE- and MMP-inhibition. Cardiovasc Res 46: 298-306, 2000; (30) Lim H H, DeLano F A, and Schmid-Schonbein G W. Life and death cell labeling in the microcirculation of the spontaneously hypertensive rat. J Vasc Res 38: 228-236, 2001; (31) Lip G Y, Blann A D, Zarifis J, Beevers M, Lip P L, and Beevers D G. Soluble adhesion molecule P-selectin and endothelial dysfunction in essential Hypertension: implications for atherogenesis? A preliminary report. Journal of Hypertension 13: 1674-1678, 1995; (32) Morisco C, Condorelli G, Orzi F, Vigliotta G, Di Grezia R, Beguinot F, Trimarco B, and Lembo G. Insulin-stimulated cardiac glucose uptake is impaired in spontaneously hypertensive rats: role of early steps of insulin signaling. Journal of Hypertension 18: 465473, 2000; (33) Mujumdar V S, Smiley L M, and Tyagi S C. Activation of matrix metalloproteinase dilates and decreases cardiac tensile strength. Int J Cardiol 79: 277-286, 2001; (34) Newaz M A, Yousefipour Z, and Nawal N N. Modulation of nitric oxide synthase activity in brain, liver, and blood vessels of spontaneously hypertensive rats by ascorbic acid: protection from free radical injury. Clin Exp Hypertens 27: 497-508, 2005; (35) Newman E C and Frank C W. Circular dichroism spectra of tetracycline complexes with Mg+2 and Ca+2. J Pharm Sci 65: 1728-1732, 1976; (36) Peterson J T, Hallak H, Johnson L, Li H, O'Brien P M, Sliskovic D R, Bocan T M, Coker M L, Etoh T, and Spinale F G. Matrix metalloproteinase inhibition attenuates left ventricular remodeling and dysfunction in a rat model of progressive heart failure. Circulation 103: 2303-2309, 2001; (37) Rajagopalan S, Meng X P, Ramasamy S, Harrison D G, and Galis Z S. Reactive oxygen species produced by macrophage-derived foam cells regulate the activity of vascular matrix metalloproteinases in vitro. Implications for atherosclerotic plaque stability. J Clin Invest 98: 2572-2579, 1996; (38) Rosenberg G A, Navratil M, Barone F, and Feuerstein G. Proteolytic cascade enzymes increase in focal cerebral ischemia in rat. J Cereb Blood Flow Metab 16: 360-366, 1996; (39) Ryan M E, Ramamurthy N S, and Golub L M. Tetracyclines inhibit protein glycation in experimental Diabetes. Adv Dent Res 12: 152-158, 1998; (40) Schmid-Schönbein G W, Seiffge D, DeLano F A, Shen K, and Zweifach B W. Leukocyte counts and activation in spontaneously hypertensive and normotensive rats. Hypertension 17: 323-330, 1991; (41) Seccia T M, Bettini E, Vulpis V, Quartaroli M, Trist D G, Gaviraghi G, and Pirrelli A. Extracellular matrix gene expression in the left ventricular tissue of spontaneously hypertensive rats. Blood Press 8: 5764, 1999; (42) Sechi L A, Griffin C A, Giacchetti G, Zingaro L, Catena C, Bartoli E, and Schambelan M. Abnormalities of insulin receptors in spontaneously hypertensive rats. Hypertension 27: 955-961, 1996; (43) Shen K, DeLano F A, Zweifach B W, and Schmid-Schoenbein G W. Circulating leukocyte counts, activation, and degranulation in Dahl hypertensive rats. Circ Res 76: 276-283, 1995; (44) Shen K, Sung K L, Whittemore D E, DeLano F A, Zweifach B W, and Schmid-Schonbein G W. Properties of circulating leukocytes in spontaneously hypertensive rats. Biochem Cell Biol 73: 491-500, 1995; (45) Sironi L, Maria Calvio A, Bellosta S, Lodetti B, Guerrini U, Monetti M, Tremoli E, and Mussoni L. Endogenous proteolytic activity in a rat model of spontaneous cerebral stroke. Brain Res 974: 184-192, 2003; (46) Sorsa T, Lindy O, Konttinen Y T, Suomalainen K, Ingman T. Saari H, Halinen S, Lee H M, Golub L M, Hall J, and et al. Doxycycline in the protection of serum alpha-1-antitrypsin from human neutrophil collagenase and gelatinase. Antimicrob Agents Chemother 37: 592-594, 1993; (47) Spiers J P, Kelso E J, Siah W F, Edge G, Song G, McDermott B J, and Hennessy M. Alterations in vascular matrix metalloproteinase due to ageing and chronic Hypertension: effects of endothelin receptor blockade. J Hypertens 23: 1717-1724, 2005; (48) Spinale F G. Matrix metalloproteinases: regulation and dysregulation in the failing heart. Circ Res 90: 520-530, 2002; (49) Suematsu M, Suzuki H. Delano F A, and Schmid-Schonbein G W. The inflammatory aspect of the microcirculation in Hypertension: oxidative stress, leukocytes/endothelial interaction, apoptosis. Microcirculation 9: 259-276, 2002; (50) Suematsu M, Suzuki H. Tamatani T, Iigou Y, DeLano F A, Miyasaka M, Forrest M J, Kannagi R, Zweifach B W, Ishimura Y, and Schmid-Schönbein G W. Impairment of selectin-mediated leukocyte adhesion to venular endothelium in spontaneously hypertensive rats. J Clin Invest 96: 2009-2016, 1995; (51) Sumii T and Lo E H. Involvement of matrix metalloproteinase in thrombolysis-associated hemorrhagic transformation after embolic focal ischemia in rats. Stroke 33: 831-836, 2002; (52) Suzuki H, Schmid-Schonbein G W, Suematsu M, DeLano F A, Forrest M J, Miyasaka M, and Zweifach B W. Impaired leukocyte-endothelial cell interaction in spontaneously hypertensive rats. Hypertension 24: 719-727, 1994; (53) Suzuki H, Zweifach B W, Forrest M J, and Schmid-Schönbein G W. Modification of leukocyte adhesion in spontaneously hypertensive rats by adrenal corticosteroids. J Leukoc Biol 57: 20-26, 1995; (54) Takase S, Lerond L, Bergan J J, and Schmid-Schonbein G W. The inflammatory reaction during venous Hypertension in the rat. Microcirculation 7: 41-52, 2000; (55) Touyz R M. Reactive oxygen species, vascular oxidative stress, and redox signaling in Hypertension: what is the clinical significance? Hypertension 44: 248-252, 2004; (56) Uitto V J, Firth J D, Nip L, and Golub L M. Doxycycline and chemically modified tetracyclines inhibit gelatinase A (MMP-2) gene expression in human skin keratinocytes. Ann N Y Acad Sci 732: 140-151, 1994; (57) Yamada E, Hazama F, Kataoka H, Amano S, Sasahara M, Kayembe K, and Katayama K. Elastase-like enzyme in the aorta of spontaneously hypertensive rats. Virchows Arch B Cell Pathol Incl Mol Pathol 44: 241-245, 1983; (58) Yasmin, McEniery C M, Wallace S. Dakham Z, Pulsalkar P, Maki-Petaja K, Ashby M J, Cockcroft J R, and Wilkinson I B. Matrix metalloproteinase-9 (MMP-9), MMP-2, and serum elastase activity are associated with systolic Hypertension and arterial stiffness. Arterioscier Thromb Vasc Biol 25: 372, 2005; (59) Zweifach B W, Kovalcheck S, DeLano F A, and Chen P. Micropressure-flow relationship in a skeletal muscle of spontaneously hypertensive rats. Hypertension 3: 601-614, 1981; (60) Kobayashi, N., DeLano, F. A., Schmid-Schönbein, G. W.: Oxidative stress promotes endothelial cell apoptosis and loss of microvessels in the spontaneous hypertensive rats. Arteriosclerosis, Thrombosis and Vascular Biology 25: 2114-2121, 2005.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

In describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A compound for treating inflammatory pathologies and syndrome, the compound comprising:
an effective dose of an MMP inhibitor, resulting in decreased symptoms of inflammatory pathologies and syndrome.

2. The compound of claim 1, wherein the pathologies and syndrome are selected from the group consisting of: insulin resistance, hypertension, thrombosis, and free radical production.

3. The compound of claim 1, wherein the effective dose is provided orally.

4. The compound of claim 1, wherein the MMP inhibitor is synthetic.

5. The compound of claim 1, wherein the MMP inhibitor comprises doxycycline.

6. The compound of claim 1, wherein the MMP inhibitor is naturally occurring.

7. The compound of claim 1, wherein the MMP inhibitor is added to drinking water.

8. The compound of claim 1, wherein the compound lowers pancreatic proteinase activity.

9. A compound to treat inflammatory pathologies and syndrome, the compound comprising:
an effective dose of an MMP inhibitor, resulting in lowered blood pressure and lowered plasma and tissue proteinase activity related to inflammatory pathologies and syndrome.

10. A method of treating inflammation, the method comprising:
providing an effective dose of an MMP inhibitor to treat inflammation, resulting in lowered hypertension, reduced receptor cleavage, normalization of plasma glucose levels, reduced apoptosis and capillary rarefaction, and restoration of leukocyte adhesion molecule expression on leukocytes.

11. The method of claim 10, wherein the effective dose is provided orally.

12. The method of claim 10, wherein the MMP inhibitor comprises doxycycline.

13. The method of claim 10, wherein the MMP inhibitor is provided by inclusion in drinking water.

14. A method of treating syndrome x, the method comprising:
providing an effective dose of an MMP inhibitor to treat metabolic syndrome x, resulting in decreased microvascular proteinase activity.

15. The method of claim 14, wherein the effective dose is provided orally.

16. The method of claim 14, wherein MMP inhibitor comprises doxycycline.

17. The method of claim 14, wherein the MMP inhibitor is provided by inclusion in drinking water.

18. A method for treating disease, the method comprising:
exposing abnormally functioning cells to an effective dose of a substance that decreases the amount of MMPs present within their cytoplasm and membranes.

19. The method of claim 18, wherein the white blood cells are granular leukocytes.

20. The method of claim 19, wherein the granular leukocytes are neutrophils.

21. The method of claim 18, wherein the substance is an MMP-inhibitor.

22. The method of claim 21, wherein the MMP-inhibitor is doxycycline.

23. A method of treating disease, the method comprising:
exposing cells to an effective dose of a substance that is capable of decreasing blood plasma protease activity, reducing oxygen free radical formation, and inhibiting cleavage of membrane receptors.

24. The method of claim 23, wherein the substance is composed of doxycycline.

25. The method of claim 23, wherein the membrane receptors are the insulin receptor α and the CD18 integrin.

26. A method for treating disease, the method comprising:
introducing about 1.0-20.0 mg/kg/day of an MMP inhibitor daily into a patient's circulatory system for a defined period of time.

27. The method of claim 26, wherein the MMP inhibitor is doxycycline.

28. The method of claim 26, wherein the introduction is performed via an oral ingestion of a solution containing the MMP inhibitor.

29. The method of claim 26, wherein the defined period of time is no longer than 24 weeks.

30. The methods of claim 26, wherein the defined period of time is continuous in genetic forms of hypertension or in patients with metabolic X syndrome.

31. A composition comprising an effective dose of a substance dissolved in fluid to produce a solution, said solution capable of inhibiting MMP activity in neutrophils.

32. The composition of claim 31, wherein the substance is doxycycline.

33. A container containing the composition of claim 31.

34. A kit comprising at least one container of claim 33 containing a substance that inhibits or decreases MMP activity in cells.

35. The kit of claim 34, wherein the substance is composed of doxycycline and the cells are white blood cells.

36. The kit of claim 35, wherein the white blood cells are neutrophils.

37. A method for treating hypertension, the method comprising:
dissolving between 5.5-220 mg of an MMP-inhibitor solute into a liter of fluid to produce a solution; and
consuming about 1.0-20.0 mg/kg/day of the resultant solution for a period of time effective to affect hypertension.

38. The method of claim 37, wherein the MMP-inhibitor solute comprises doxycycline.

39. The method of claim 37, wherein the mass of the MMP-inhibitor solute is between 75-180 mg.

40. The method of claim 39, wherein the mass of the MMP-inhibitor solute is between 100-150 mg.
